(19) United States
(12) Patent Application Publication
(10) Pub. No.: US 2012/0027729 A1
(43) Pub. Date: Feb. 2, 2012

(54) METHODS FOR TREATING DIABETES

(75) Inventors: Darwin J. Prockop, Philadelphia, PA (US); Ryang Hwa Lee, Tempe, TX (US); Min Jeong Seo, Busan (KR)

(73) Assignee: Tulane University Health Sciences Center, New Orleans, LA (US)

(21) Appl. No.: 12/298,606

(22) PCT Filed: Apr. 27, 2007
(86) PCT No.: PCT/US07/10309

§ 371 Date: Aug. 11, 2011

Related U.S. Application Data
(60) Provisional application No. 60/795,889, filed on Apr. 28, 2006.
(60) Provisional application No. 60/852,027, filed on Oct. 16, 2006.

(51) Int. Cl. A61K 35/12; A61P 3/10

(52) U.S. Cl. 424/93.7

(57) ABSTRACT

Transplantation of multipotent stromal cells (MSCs) into diabetic mice lowers blood sugar, increases blood insulin levels, increases the number and size of islets, and improves renal pathology. Accordingly, the invention provides methods for treating or preventing diabetes by administering isolated MSCs. The invention also provides methods for treating or preventing complications which arise from diabetes, including diabetic nephropathy, by transplanting isolated MSCs.

This application claims priority to U.S. Provisional Application Nos. 60/795,889, filed Apr. 28, 2006 and 60/852,027, filed Oct. 16, 2006, the contents of which are incorporated herein by reference.

The present invention was made in part with support from grants obtained from the National Institutes of Health. The federal government may have rights in the present invention.

### FIELD OF THE INVENTION

The present invention generally relates to the therapeutic uses of multipotent stromal cells in the treatment of diabetes and complications of diabetes, including nephropathy.

### BACKGROUND OF THE INVENTION

Diabetes refers to a disease process characterized by elevated levels of plasma glucose or hyperglycemia in the fasting state or after administration of glucose during an oral glucose tolerance test. Persistent or uncontrolled hyperglycemia is associated with increased and premature morbidity and mortality. Often abnormal glucose homeostasis is associated both directly and indirectly with alterations of the lipid, lipoprotein and apolipoprotein metabolism and other metabolic and hemodynamic disease. Therefore patients with diabetes mellitus are at especially increased risk of macrovascular and microvascular complications, including coronary heart disease, stroke, peripheral vascular disease, hypertension, nephropathy, neuropathy, and retinopathy.

There are two generally recognized forms of diabetes. In type 1 diabetes, or insulin-dependent diabetes mellitus (IDDM), patients produce little or no insulin, the hormone which regulates glucose utilization. In type 2 diabetes; or noninsulin dependent diabetes mellitus (NIDDM), patients often have plasma insulin levels that are the same or even elevated compared to nondiabetic subjects; however, these patients have developed a resistance to the insulin stimulating effect on glucose and lipid metabolism in the main insulin-sensitive tissues, which are muscle, liver and adipose tissues, and the plasma insulin levels, while elevated, are insufficient to overcome the pronounced insulin resistance. Insulin resistance is not primarily due to a diminished number of insulin receptors, but is due to a post-insulin receptor binding defect that is not yet fully understood. This resistance to insulin responsiveness results in insufficient insulin activation of glucose uptake, oxidation and storage in muscle and inadequate insulin repression of lipolysis in adipose tissue and of glucose production and secretion in the liver.

The abnormally high blood glucose (hyperglycemia) that characterizes both type 1 and type 2 diabetes, if left untreated, results in a variety of pathological conditions, including premature blindness, nerve damage, cardiovascular disease, stroke, and kidney failure (Sheetz and King, JAMA 288:2579-2588 (2002)). For example, diabetic nephropathy is a major long-term complication of diabetes mellitus, and is the leading indication for dialysis and kidney transplantation in the United States (Marks and Raskin, Med. Clin. North Am. 82:877-907 (1998)). The development of diabetic nephropathy is seen in 25 to 50% of type 1 and type 2 diabetic patients. Accordingly, diabetic nephropathy is the most common cause of end-stage renal disease and kidney failure in the Western world.

A potential treatment for diabetes would be to restore β cell function so that insulin release is dynamically regulated in response to changes in blood glucose levels. This can be achieved by pancreas transplantation, but this approach is typically limited to diabetics requiring kidney transplants for renal failure. Also, pancreas transplantation can require life-long immunosuppression to prevent allogeneic graft rejection and autoimmune destruction of the transplanted pancreas.

Recently, transplants of isolated human islet preparations have successfully reversed insulin dependent diabetes in human subjects for prolonged periods. However, a large amount of donor islet cell material is required for each recipient, and the supply of islet cell material has not been sufficient to meet the demand.

The shortage of donor islets has prompted research into alternative sources of glucose-responsive, insulin-producing cells, including the potential of using stem/progenitor cells. Although promising results have been reported with embryonic stem cells of rodent origin (see, e.g., Hori et al., Proc. Natl. Acad. Sci. U.S.A. 99:16105-10 (2002); Lumelsky et al., Science 292:1389-97 (2001)), the potential use of human embryonic stem cells to treat human diseases is scientifically uncertain at this time, in large part because of the tendency of embryonic stem cells to produce tumors, as was seen in diabetic mice (Fujikawa et al., Am. J. Pathtol. 166:1781-1791 (2005)). As a result, several groups have studied various potential sources of adult pancreatic stem/progenitor cells.

Bone marrow-derived stem or progenitor cells are an attractive source for generating cells useful for transplantation into diabetes patients. Bone marrow is readily accessible for isolating stem cells, and bone marrow transplants have been used to treat patients with leukemias and other disorders for more than thirty years. In addition, unlike other organs, bone marrow cells can be frozen for prolonged time periods (cryopreserved) without damaging too many cells.

Studies of bone marrow transplantation to treat diabetes have focused on restoring β cell function, and have presented conflicting observations as to whether or not cells from bone marrow can be a potential therapy of diabetes mellitus.

One approach has been to use whole, unfractionated bone marrow. In this approach, whole bone marrow is genetically labeled prior to transplantation, and labeled insulin-producing cells are identified in the recipient mice. One study using a CRE-LoxP-GFP system found that 1.7 to 3% of the cells in islets of the recipient mice were marrow-derived, and that GFP-labeled donor cells isolated from the islets expressed insulin, glucose transporter 2 and transcription factors typically found in β-cells (Ianus et al., 2003). However, this report has been widely criticized in light of three subsequent reports, in which mice were transplanted with GFP-expressing bone marrow and no evidence was found of marrow cells becoming insulin-producing cells in the pancreas of recipient mice (Choi et al., Biochem. Biophys. Res. Commun. 330:1299-1305 (2005); Lechner et al., Diabetes 53:616-623 (2004); Taneera et al., Diabetes 55:290-296 (2006)). Bone marrow contains at least two types of stem cells. Hematopoietic stem cells (HSCs) represent the vast majority of stem cells in the bone marrow; much rarer are stem cells for non-hematopoietic tissues, variously referred to as mesenchymal stem cells or multipotent stromal cells (MSCs). The initial report of Ianus et al. suggests it is the HSCs that were found in the islets.

A second strategy has also focused specifically on the use of HSCs as well as whole bone marrow to determine whether transplanted stem cells can enhance regeneration of pancreatic insulin-producing cells in diabetic models. Hess et al. transplanted c-kit+ HSCs or whole marrow into diabetic animals with partial marrow ablation, to promote engraftment (Nat. Biotechnol. 21:763-770 (2003)). This study reported that in NOD/scid mice in which diabetes was induced with STZ, partial marrow ablation followed by transplantation of either GFP-labeled-whole marrow or GFP-labeled c-kit+ HSCs from marrow enhanced regeneration of islets, lowered blood sugar, and increased blood insulin levels. In additional experiments, multiple infusions of unfractionated whole bone marrow cells into mice with STZ-induced diabetes lowered blood sugar and improved the histomorphology of the pancreas (Banerjee et al., Biochem. Biophys. Res. Commun. 328:318-325 (2005)). In experiments in which NOD mice were used as a model for type 1 diabetes, transplantation of wild type bone marrow lowered blood sugar if the transplant was performed before, but not after, the onset of hyperglycemia (Kang et al., Exp. Hematol. 33:699-705 (2005)).

A third strategy for generating cells for transplantation has been to first differentiate marrow-derived cells into insulin-producing cells in culture, prior to transplantation. Four recent reports indicated that MSCs, identified as plastic adherent bone marrow cells, can be directed to differentiate in vitro into insulin secreting cells (Oh et al., Lab. Invest. 84:607-617 (2004)); Chen et al., World J. Gastroenterol. 10:3016-3020 (2004); Choi et al., Biochem. Biophys. Res. Commun. 330:1299-1305 (2005); and Tang et al., Diabetes 53:1721-1732 (2004)). Two of these, reports also demonstrated that transplantation of these in vitro-differentiated cells could lower blood sugar in diabetic mice (Oh et at. (2004); Tang et al. (2004)).

Given the central role pancreatic islets play in diabetes, attention in the vast majority of stem cell transplantation studies to date has focused on repair of the pancreas. However, diabetic complications, largely caused by chronic hyperglycemia, are the major cause of morbidity and mortality in diabetic patients. Few studies have addressed the effect of transplanted stem cells on non-pancreatic tissues, including the kidney, nerves, and retina. In one study, transplantation of large numbers of human umbilical cord cells into mice that were genetic models of type 2 diabetes decreased blood sugar and attenuated renal hypertrophy (Ende et al., 2004). Two other studies looked at the effect of transplanting MSCs in animal models of nephropathy, where the kidney damage was not caused by diabetes but instead was induced by either injection of an antibody or glycerol (Hauger et al., Radiology 238:200-210 (2006); Herrera et al., Int. J. Mol. Med. 14:1035-1041 (2004)).

Therefore, there exists a need to develop new therapeutic methods for treating diabetes, and complications associated with diabetes including nephropathy, neuropathy, retinopathy, stroke, and cardiovascular disease.

### SUMMARY OF THE INVENTION

Transplantation of human multipotent stromal cells (MSCs) into diabetic mice lowers blood sugar, increases blood insulin levels, increases the number and size of islets, and improves renal pathology. Accordingly, the invention provides methods for treating or preventing diabetes by administering MSCs. The invention also provides methods for treating or preventing complications which arise from diabetes, including diabetic nephropathy, by transplanting MSCs.

One embodiment of the invention provides a method of treating diabetes in an individual comprising administering to said individual a therapeutically effective amount of multipotent stromal cells. Administration of the multipotent stromal cells enhances regeneration of pancreatic islets, reduces hyperglycemia, increases insulin levels in the individual, and improves the diabetic nephropathy in the individual.

Another embodiment of the invention provides methods for preventing or inhibiting the progression of a diabetic complication in an individual by administering a therapeutically effective amount of multipotent stromal cells. Diabetic complications include microvascular complications, including diabetic nephropathy, diabetic neuropathy, and diabetic retinopathy, as well as cardiovascular disease such as stroke and heart disease.

Another embodiment of the invention provides methods for reversing hyperglycemia in an individual by administering a therapeutically effective amount of multipotent stromal cells. In one embodiment, the hyperglycemia is caused by diabetes.

Another embodiment of the invention provides methods for reversing hypoinsulinemia in an individual by administering a therapeutically effective amount of multipotent stromal cells. In one embodiment, the hypoinsulinemia is caused by pancreatic damage, including damage caused by diabetes.

Another embodiment of the invention provides methods for enhancing the regeneration or repair of pancreatic islets in an individual by administering a therapeutically effective amount of multipotent stromal cells.

Multipotent stromal cells can be isolated from tissues including bone marrow, peripheral blood, umbilical cord blood, and synovial membrane. In one preferred embodiment the multipotent stromal cells are isolated from bone marrow. Prior to administration, the multipotent stromal cells can be cultured in vitro. In one preferred embodiment, the multipotent stromal cells are expanded in vitro prior to administration to the individual.

Multipotent stromal cells for administration can be isolated from the individual to be treated, i.e. autologous, or isolated from another individual, i.e. allogeneic. For allogeneic multipotent stromal cells, it is preferred that the donor and the individual to be treated are HLA compatible. Multipotent stromal cells can be isolated from a mammal, including a rodent, a horse, a cow, a pig, a dog, a cat, a non-human primate, and a human. Human multipotent stromal cells are preferred in certain embodiments.

The individual to be treated with the multipotent stromal cells can be a mammal, including a rodent, a horse, a cow, a pig, a dog, a cat, a non-human primate, and a human. In certain preferred embodiments, the mammal is a human.

The multipotent stromal cells can be administered by infusion, including intravenous infusion, systemic infusion, intra-arterial infusion, intracoronary infusion, and intracardiac infusion.

One embodiment of the invention provides the use of isolated multipotent stromal cells for treating diabetes in an individual in need thereof. Another embodiment provides the use of isolated multipotent stromal cells in the manufacture of a medicament for treating diabetes in an individual in need thereof.

### BRIEF DESCRIPTION OF THE DRAWINGS

FIGS. 1A-1C show the effects of hMSCs on blood glucose and mouse insulin levels in STZ-induced diabetic NOD/scidSCID mice. The experimental design is shown in the top panel. FIG. 1A shows blood glucose levels in untreated diabetic mice (STZ-treated mice) and in hMSC-treated diabetic mice (STZ-treated mice+hMSCs). Values are mean+/−S.E. FIG. 1B shows blood glucose levels in untreated diabetic mice and diabetic mice infused with human fibroblasts (STZ-treated mice+hFibroblasts). Differences on Day 10 reflect variations in untreated mice before fibroblasts were infused. Values are mean+/−S.D. FIG. 1C shows blood levels of mouse insulin on Day 32 in diabetic mice (STZ), hMSC-treated diabetic mice (STZ+hMSCs) and normal mice. Values are mean+/−S.D. Asterisks indicate values that differ with p=0.0018.

FIGS. 2A-2D show the results of immunohistochemistry of pancreas from diabetic mice (STZ-treated), hMSC-treated diabetic mice (STZ+hMSCs), and control mice (Normal) at Day 32. FIG. 2A is a photomicrograph that shows the morphology of islets stained with hematoxylin and eosin. Sections of 5 μm magnified ×400. FIG. 2B is a series of photomicrographs that show islets labeled with antibodies for mouse insulin; nuclei were labeled with DAPI. Sections of 5 μm magnified ×400. FIG. 2C is a graph that shows the number of insulin pixels per islet. Values are mean+/−S.D. Asterisks indicate values that differ with p=0.0079. FIG. 2D is a graph that shows the number of islets per section. Values are mean+/−S.D. Asterisks indicate values that differ with p=0.002; n=4 or 5.

FIG. 3 is a series of photomicrographs that show immunohistochemistry of pancreas from hMSC-treated diabetic NOD/scid mice on Day 32. Sections were co-labeled with antibodies for human cells (β2-microglobulin) and mouse insulin; nuclei were stained with DAPI. 5 μm sections are magnified ×400. The dotted lines indicate outlines of ducts; arrows indicate human cells; arrowheads indicate human cells co-labeled for mouse insulin.

FIGS. 4A-4C show renal glomeruli from diabetic mice (STZ), hMSC-treated diabetic mice (STZ+hMSCs) and normal mice on Day 32. FIG. 4A shows photomicrographs of glomeruli stained with Periodic Acid Schiff. 8 μm sections are magnified ×400. In FIG. 4B, glomeruli were labeled with antibodies to mouse macrophages/monocytes. 8 μm sections are magnified ×400. FIG. 4C is a graph that shows the number of pixels per glomerulus in sections labeled with antibodies to mouse macrophages/monocytes. Values are mean+/−S.D. Asterisks indicate values that differ with p<0.005.

FIGS. 5A-5L are photomicrographs that show renal glomeruli from hMSC-treated diabetic mice on Day 32. 5 μm sections are magnified ×400. FIGS. 5A-5D show glomeruli labeled with antibodies for human nuclei antigen and mouse/human fibronectin. Some human cells that are co-labeled have the rounded morphology of mesangial cells. FIGS. 5E-5H show glomeruli labeled with antibodies for human nuclei antigen and mouse/human podocalyxin. No co-labeling was detected. FIGS. 5I-5L show deconvolution images of glomeruli labeled for human nuclei antigen and a marker for mouse/human endothelial cells, CD31. Some human cells appear to be co-labeled and have the elongated morphology of endothelial cells. Arrows: human cells. Dotted arrows indicate the planes for deconvolution; dotted lines indicate outlines of glomeruli. Additional de-convoluted images are shown in FIGS. 7, 8, and 9.

FIGS. 6A-6B show results from analysis of pancreas from hMSC-treated diabetic mice on Day 32. FIG. 6A is a series of photomicrographs of pancreas sections that show co-labeling for human cells (β2-microglobulin) and PDX-1 or human insulin. Nuclei are labeled with DAPI; 5 μm sections are magnified ×400. Arrows indicate human cells co-labeled with mouse/human PDX-1 or human insulin. FIG. 6B shows RT-PCR assays for human insulin mRNA isolated from pancreas. The cDNA has the predicted size of 245 bp and is cleaved into fragments of the predicted size by SbfI and EcoNI. RT-PCR assays for human insulin mRNA in 11 other hMSC-treated diabetic mice were negative.

FIG. 7 shows photomicrographs of kidney from hMSC-treated diabetic mice. The sample was co-labeled with antibodies to human nuclei antigen and mouse/human CD31. Nuclei were labeled with DAPI. 10 μm sections are magnified ×400. The inserts are enlargements of glomeruli labeled with human nuclei antigen and stained with DAPI.

FIG. 8 shows photomicrographs of three-dimensional deconvolutional microscopy of glomeruli from hMSC-treated diabetic mice. Sections were co-labeled with antibodies to human nuclei antigen and mouse/human CD31. 10, 20 or 30 μm sections are magnified ×400. Arrows indicate the planes of deconvoluted images.

### DETAILED DESCRIPTION

We have now discovered that administration of human multipotent stem cells in an animal model of diabetes enhances regeneration of pancreatic islets, reduces hyperglycemia, increases insulin levels in the individual, and improves the diabetic nephropathy in the individual. Accordingly, the invention provides cell-based therapies for the treatment of diabetes and complications of diabetes by administering to an individual a therapeutically effective amount of mesenchymal stem cells.

### I. Cell-Based Therapies

One embodiment of the invention provides a method of treating diabetes in an individual comprising administering to said individual a therapeutically effective amount of MSCs.

As used herein, “diabetes” includes diabetes mellitus type 1 and diabetes mellitus type 2, as well as early stage diabetes and a pre-diabetic condition characterized by mildly decreased insulin or mildly elevated blood glucose levels. “Diabetes mellitus type 1” refers to insulin-dependent diabetes mellitus, and “diabetes mellitus type 2” refers to non-insulin dependent diabetes mellitus. The symptoms of diabetes mellitus type 1 include hyperglycemia, glycosuria, deficiency of insulin, polyuria, polydypsia; and/or ketonuria. The symptoms of diabetes mellitus type 2 include those of type 1 as well as insulin resistance.

The methods of the invention can be used to treat type 1 diabetes patients by increasing the number and size of pancreatic islets, thereby replacing lost pancreatic β cells. The methods of the invention can also be used to treat type 2 diabetes patients by increasing the number and size of pancreatic islets, thereby increasing insulin production.

In the methods of the invention, MSCs are administered to the patient afflicted with diabetes in an amount sufficient to provide an effective level of endogenous insulin in the patient. An “effective” or “normal” level of endogenous insulin in a patient refers generally to the level of insulin that is produced endogenously in a healthy patient, i.e., a patient who is not afflicted with diabetes. Alternatively, an “effective” level may also refer to the level of insulin that is determined by the practitioner to be medically effective to alleviate the symptoms of diabetes.

A number of different endpoints can be used to determine whether the administration of MSCs improves the diabetes or associated conditions in the individual. For example, transplantation of MSCs can increase the functional mass of β cells in the pancreatic islets. Other endpoints include measurement of enhanced plasma levels of circulating C peptide and insulin after injecting mice with β cell stimulants such as glucose or arginine; a response to gastrin/EGF treatment demonstrated by increased insulin immunoreactivity or mRNA levels extracted from the islet transplants; and increased number of β cells, determined by morphometric measurement of islets in treated individuals.

Another embodiment of the invention provides methods for preventing or inhibiting the progression of a diabetic complication in an individual by administering a therapeutically effective amount of MSCs. Diabetic complications include microvascular complications, including diabetic nephropathy, diabetic neuropathy, and diabetic retinopathy, as well as cardiovascular disease such as stroke and heart disease. Other complications from diabetes include but are not limited to macroangiopathy, obesity, hyperinsulinemia, sugar metabolism disorders, hyperlipemia, hypercholesteremia, hypertriglyceridemia, lipid metabolism disorders, edema, hyperuricemia, and gout.

In one embodiment of the invention, transplantation of MSCs is used to treat or prevent diabetic nephropathy. Contributing risk factors associated with the development of diabetic nephropathy and other renal disorders in subjects with type 1 or type 2 diabetes include hyperglycemia, hypertension, altered glomerular hemodynamics, and increased or aberrant expression of various growth factors, including transforming growth factor-beta (TGF-β), insulin-like growth factor (IGF)-I, vascular endothelial growth factor-a (VEGF-A), and connective tissue growth factor (CTGF). See, e.g., Flyvbjerg, Diabetologia 43:1205-23 (2000); Brosius, Exp. Diab. Res. 4:225-233 (2003); Gilbert et al., Diabetes Care 26:2632-2636 (2003); and International Publication No. WO 00/13706.

The transplantation of MSCs to treat diabetic nephropathy can be used in combination with any other regimes for treating nephropathy. Current treatment strategies directed at slowing the progression of diabetic nephropathy use various approaches, including optimized glycemic control through modification of diet and/or insulin therapy and hypertension control, have demonstrated varying degrees of success. For example, both angiotensin-converting enzyme (ACE) inhibitors and angiotensin receptor blockers (ARBs), administered to reduce hypertension, have been shown to delay progression or development of nephropathy and macroalbuminuria.

Another embodiment of the invention provides methods for treating and/or reversing hyperglycemia in an individual by administering a therapeutically effective amount of multipotent stromal cells. In one embodiment, the hyperglycemia is caused by diabetes.

Disorders caused by hyperglycemia include diabetic complications such as retinopathy, neuropathy, nephropathy, ulcers, and macroangiopathy; obesity; hyperinsulinemia; disorders of sugar metabolism; hyperlipemia; hypercholesteremia; hypertriglyceridemia; disorders of lipid metabolism; atherosclerotic cardiovascular disease; hypertension; congestive failure; edema; hyperuricemia and gout.

The term “treating hyperglycemia” means that glucose levels in the treated individual are reduced as compared to the glucose levels in that individual in the absence of treatment. Glucose levels can be measured using techniques known in the art. For example, blood glucose levels can be measured with the glucometer such as the Elite® diabetes care system (Bayer, Germany).

Another embodiment of the invention provides methods for treating and/or reversing hypoinsulinemia in an individual by administering a therapeutically effective amount of MSCs. In one embodiment, the hypoinsulinemia is caused by pancreatic damage, including damage caused by diabetes.

“Hypoinsulinemia” is a condition characterized by lower than normal amounts of insulin circulating throughout the body. Obesity is generally not involved. This condition includes type 1 diabetes.

The term “treating hypoinsulinemia” means that insulin levels in the treated individual are increased as compared to insulin levels in that individual in the absence of treatment. Insulin levels can be measured using techniques known in the art, including measuring circulating insulin levels, sometimes referred to as serum insulin levels, as well as pancreatic insulin levels.

Another embodiment of the invention provides methods for enhancing the regeneration or repair of pancreatic islets in an individual by administering a therapeutically effective amount of MSCs.

To assess the regeneration of pancreatic islets in an individual, the size and function of newly developed β insulin secreting cells or islets can be measured using standard physiological or diagnostic parameters, including any of the following: islet β cell mass, islet β cell number, islet β cell percent, blood glucose, serum glucose, blood glycosylated hemoglobin, pancreatic β cell mass, pancreatic β cell number, fasting plasma C peptide content, serum insulin, and/or pancreatic insulin content.

Methods of the invention which provide treatments for diabetes that result in relief of its symptoms can be tested in an animal which exhibits symptoms of diabetes, such that the animal will serve as a model for methods and procedures useful in treating diabetes in humans. Potential treatments for diabetes can therefore be first examined in the animal model by administering the potential treatment to the animal and observing the effects, comparing the treated animals to untreated controls.

One important model of type 1 or insulin dependent diabetes which is a particularly relevant model for human diabetes is the non-obese diabetic (NOD) mouse (Kikutano and Makino, Adv. Immunol. 52:285 (1992) and references cited therein). The development of type 1 diabetes in NOD mice occurs spontaneously and suddenly, without any external stimuli. As NOD mice develop diabetes, they undergo a progressive destruction of β cells which is caused by a chronic autoimmune disease. The development of insulin-dependent diabetes mellitus in NOD mice can be divided roughly into two phases: initiation of autoimmune insulitis (lymphocytic inflammation in the pancreatic islets) and promotion of islet destruction and overt diabetes. Diabetic NOD mice begin life with euglycemia, or normal blood glucose levels, but by about 15 to 16 weeks of age the NOD mice start becoming hyperglycemic, indicating the destruction of the majority of their pancreatic β cells and the corresponding inability of the pancreas to produce sufficient insulin. In addition to insulin deficiency and hyperglycemia, diabetic NOD mice experience severe glycosuria, polydypsia, and polyuria, accompanied by a rapid weight loss (Kikutano and Makino, 1992). Thus, both the cause and the progression of the disease are similar to human patients afflicted with type 1 diabetes. Spontaneous remission is rarely observed in NOD mice, and these diabetic animals die one to two months after the onset of diabetes unless they receive insulin therapy. Accordingly, the NOD mouse can be used as an animal model to test the effectiveness of the various methods of treatment of diabetes by administering MSCs.

The effectiveness of the treatment methods of the invention on diabetes in the NOD mice can be monitored by assaying for diabetes in the NOD mice by means known to those of skill in the art, including examining the NOD mice for polydipsia, polyuria, glycosuria, hyperglycemia, and insulin deficiency, as well as weight loss. For example, the level of urine glucose (glycosuria) can be monitored with Testape (Eli Lilly, Indianapolis, Ind.) and plasma glucose levels can be monitored with a Glucometer 3 Blood Glucose Meter (Miles, Inc., Elkhart, Ind.) as described in U.S. Pat. No. 5,888,507, incorporated herein by reference. Monitoring urine glucose and plasma glucose levels by these methods, NOD mice are considered diabetic after two consecutive urine positive tests with Testape values of +1 or higher or plasma glucose levels >250 mg/dL (U.S. Pat. No. 5,888,507).

Another means of assaying diabetes in NOD mice is to examine pancreatic insulin levels. Pancreatic insulin levels can be determined, for example, by immunoassay, and compared among treated and control mice (U.S. Pat. No. 5,470,873, incorporated herein by reference). In this case, insulin is extracted from mouse pancreas and its concentration is determined by its immunoreactivity, such as by radioimmunoassay techniques, using mouse insulin as a standard (U.S. Pat. No. 5,888,507).

A number of animal models are useful for studying type 2 or non-insulin-dependent diabetes, including the following rodent models: the Zucker Diabetic Fatty (ZDF) rat, the Wistar-Kyoto rat, the diabetes (db) mouse, and the obese (ob) mouse (Pickup and Williams, eds, Textbook of Diabetes, 2nd. Edition, Blackwell Science).

The ZDF rat is widely used an animal model of type 2 diabetes, as it displays numerous diabetic characteristics that are similar to those found in human patients with type 2 diabetes (Clark et al., Proc. Soc. Exp. Biol. Med. 173:68 (1983)). These diabetic characteristics include insulin resistance, impaired glucose tolerance, hyperglycemia, obesity, hyperinsulinemia, hyperlipidemia, and moderate hypertension. The diabetes of ZDF rats is genetically conferred and linked to the autosomal recessive fatty (fa) gene, such that ZDF rats are homozygous (fa/fa) for the fatty gene. ZDF rats typically develop the symptoms of diabetes between approximately 8-10 weeks of age, during which time β cell failure and progression to overt diabetes occurs.

The effectiveness of the treatment methods of the invention on diabetes in the ZDF rats can be monitored by assaying for diabetes in the ZDF rats by means known to those of skill in the art, including examining the ZDF rats for plasma glucose levels, plasma insulin levels, and weight gain. Plasma glucose levels are typically checked 1-2 times per week, and can be monitored with a Glucometer 3 Blood Glucose Meter (Miles, Inc., Elkhart; Ind.). Monitoring non-fasting plasma glucose levels by this methods, ZDF rats are considered diabetic when plasma glucose levels remain high (>250 mg/dL) or further increase, while effective treatment will cause rats to be non-diabetic, evidenced by a decrease in plasma glucose level (approximately 100-200 mg/dL) that is maintained (Yakubu-Madus et al., Diabetes 48:1093 (1999)).

Non-fasting insulin levels can be monitored with a commercial radioimmunoassay kit (Diagnostic Products, Los Angeles, Calif.) with porcine and rat insulin as the standards (Yakubu-Madus, 1999). In this assay, plasma insulin is monitored once per week and will remain at or above the starting level if treatment is effective against diabetes, but will decrease approximately 2-3 fold over four weeks in ZDF rats that remain diabetic.

Fasting plasma glucose and insulin levels can be determined by performing an oral glucose tolerance test (OGTT) on rats that have been fasted overnight. In a typical OGTT, rats are given 2 g glucose/kg body weight by stomach gavage, and blood samples are collected at 0, 10, 30, 60, 90, and 120 minutes Yakubu-Madus (1999). In diabetic ZDF rats, fasting glucose values will increase from approximately 100-200 mg/dl at time zero to about 400-500 mg/dl at 30-60 minutes, and then decrease to about 350-450 mg/dl by 120 minutes. If diabetes is alleviated, fasting glucose plasma values will have a lesser initial decrease to about 200-250 mg/dl at 30-60 minutes, and then decrease to about 100-150 mg/dl by 120 minutes. Plasma insulin levels measured before and during an OGTT in fasting ZDF rats will typically double in value by 10 minutes, and then decrease back to the starting value for the remainder of the assay. In contrast, effective treatment of diabetes in ZDF rats is evidenced by a 4-5 fold increase in plasma insulin at 10 minutes, followed by a linear decrease to about the starting value at 90 minutes (Yakubu-Madus, 1999).

Another means of assaying diabetes in ZDF rats is to examine pancreatic insulin levels in ZDF rats. For example, pancreatic insulin levels can be examined by immunoassay and compared among treated and control rats, as described above for the NOD mouse animal model of diabetes.

### II. Multipotent Stromal Cells (MSCS)

Bone marrow contains at least two types of stem cells, hematopoietic stem cells (HSCs) and stem cells for non-hematopoietic tissues, referred to here as multipotent stromal cells (MSCs). These plastic adherent stem/progenitor cells isolated from bone marrow were initially referred to as fibroblastoid colony forming units, then in the hematological literature as marrow stromal cells, then as mesenchymal stem cells, and most recently as multipotent stromal cells (MSCs); these cells have also been referred to mesenchymal stem cells, bone marrow stromal cells, or simply stromal cells (see e.g. Prockop, Science 276:71-74 (1997)). MSCs are sometimes referred to as mesenchymal stem cells because they are capable of differentiating into multiple mesodermal tissues, including bone (Beresford et al. (1992) J. Cell Sci. 102:341-351 (1992)), cartilage (Lennon et al., Exp. Cell Res. 219:211-222 (1995)), fat (Beresford et al., 1992) and muscle (Wakitani et al., Muscle Nerve 18:1417-1426 (1995)).

One preferred population of MSCs is a population of small and rapidly self-renewing MSCs, sometimes referred to as “RS cells” or “RS-MSCs.” RS-MSCs are described in detail in U.S. Pat. No. 7,056,738, which is incorporated herein by reference in its entirety. RS-MSCs have been demonstrated to have improved differentiation and engraftment upon transplantation into immunodeficient mice (Lee et al., Blood 107:2153-2161 (2006)).

MSCs can give rise to cells of all three germ layers, depending on conditions (Kopen et al., 1999; Liechty et al., Nature Med. 6:1282-1286 (2000); Kotton et al., Development 128:5181-5188 (2001); Toma et al., Circulation 105:93-98 (2002); Jiang et al., Nature 418:41-49 (2002)). For example, in vivo evidence indicates that unfractionated bone marrow-derived cells as well as pure populations of MSCs can give rise to epithelial cell-types including those of the lung (Krause et al., Cell 105:369-377 (2001); Petersen et al., Science 284:1168-1170 (1999)). Similarly, differentiation into neuron-like cells expressing neuronal markers has been reported (Woodbury et al., J. Neurosci. Res. 61:364-370; Sanchez-Ramos et al., Exp. Neurol. 164:247-256 (2002); Deng et al., Biochem. Biophys. Res. Commun. 282:148-152 (2001)). Under physiological conditions, MSCs are believed to maintain the architecture of bone marrow and regulate hematopoiesis with the help of different cell adhesion molecules and the secretion of cytokines, respectively (Clark et al., Ann. NY Acad. Sci. 770:70-78 (1995)).

MSCs have been used with encouraging results for transplantation in animal disease Models including osteogenesis imperfecta (Pereira et al., Proc. Nat. Acad. Sci. USA 95:1142 (1998)), parkinsonism (Schwartz et al., Hum. Gene Ther. 10:2539 (1999)), spinal cord injury (Chopp et al., Neuroreport 11:3001 (2000); Wu et al., J. Neurosci. Res. 72:393 (2003)) and cardiac disorders (Tomita et al., Circulation 100:247 (1999); Shake et al., Ann. Thorac. Surg. 73:1919 (2002)). Promising results also have been reported in clinical trials for osteogenesis imperfecta (Horwitz et al., Blood 97:1227 (2001); Horowitz et al., Proc. Natl. Acad. Sci. USA 99:8932 (2002)) and enhanced engraftment of heterologous bone marrow transplants (Frassoni et al., Int. Society for Cell Therapy SA006 (abstract) (2002); Koc et al., J. Clin. Oncol. 18:307 (2000)). Several studies have shown that engraftment of MSCs enhanced by tissue injury. Ferrari et al., Science 279:1528-1530 (1998); Okamoto et al., Nature Med. 8:1101-1017 (2002).

MSCs are easily isolated from a small aspirate of bone marrow, and readily generate single-cell derived colonies. MSCs grown out of bone marrow cell suspensions by their selective attachment to tissue culture plastic can be efficiently expanded (Azizi et al., Proc. Natl. Acad. Sci. USA 95:3908-3913 (1998); Colter et al., Proc. Natl. Acad. Sci. USA 97:3213-218 (2000)) and genetically manipulated (Schwarz et al., Hum. Gene. Ther. 10:2539-2549 (1999)).

In general, the multipotent stromal cell (MSC) therapy of the present invention involves the following steps: 1) isolation of MSCs; and 2) culture and expansion of MSCs in vitro, followed by administration of the MSCs to the individual to be treated, with or without biochemical or genetic manipulation.

### A. Isolation of MSCs

The multipotent stromal cells for use in the methods of the invention are isolated from other cells of their tissue of origin. The term “isolated” as used herein means that the cells are substantially purified from other cells, cellular components, and/or extracellular materials present in the tissue from which the MSCs are obtained. For example, bone marrow-derived MSCs are substantially purified from the other cells, such as hematopoietic stem cells, which are present in the bone marrow. The multipotent stromal cells for use in the methods of the invention are not differentiated, but remain multipotential.

Multipotent stromal cells for use in the methods of the invention can be isolated from different tissue sources, including bone marrow, peripheral blood, umbilical cord blood, and synovial membrane. Other sources of human multipotent stromal cells include, but are not limited to, embryonic yolk sac, placenta, fat, fetal and adolescent skin, and muscle tissue. In certain preferred embodiments, multipotent stromal cells can be isolated from bone marrow.

Methods for isolating MSCs for use in the methods according to the invention are known in the art. Methods for isolating MSCs from bone marrow are described for example in U.S. Pat. No. 5,486,359, as well as U.S. Patent Publication Nos. 2003/0003090, 2004/0235166, 2005/0084494, and 2004/0235165, which are incorporated herein by reference. Methods for isolating MSCs from umbilical cord blood are described in Erices et al., Br. J. Haematol. 109:235-42 (2000), which is incorporated herein by reference. Methods for isolating MSCs from synovial membrane are described for example in Djouad et al., Arthritis Res. & Ther. 7:R1304-R1315 (2005), which is incorporated herein by reference. In general, techniques for the rapid isolation of MSCs include, but not limited to, leukopheresis, density gradient fractionation, immunoselection, differential adhesion separation, and the like.

One preferred method for isolating MSCs involves collecting bone marrow aspirates, for example from the iliac crest, isolating the mononuclear cells on a density gradient, and plating the cells in culture to allow removal of non-adherent cells; the plastic-adherent cells which remain are MSCs. For example, non-adherent cells can be removed by removing the culture medium and washing the adherent cells after 24 hours in culture. This method is described in detail, for example, in U.S. Patent Publication Nos. 2003/0003090, 2004/0235166, 2005/0084494, and 2004/0235165, which are incorporated herein by reference in their entirety. Bone marrow cells may be obtained from iliac crest, femora, tibiae, spine, rib, or other medullary spaces.

One preferred method for isolating MSCs is described in detail in U.S. Pat. No. 7,056,738, which is incorporated herein by reference in its entirety. In this method, the MSCs are “RS cells,” a population of small and rapidly self-renewing MSCs. In this method, nucleated cells are isolated from bone marrow aspirates, the plastic adherent cells are isolated, and the resulting cells are plated at low density (e.g. 3 cells/cm²) and harvested before they reach confluency so that the cultures retain a special sub-population of small, spindle-shaped cells referred to as RS cells or RS-MSCs. RS-MSCs differentiate more readily and engraft more efficiently into immunodeficient mice than the larger, slowly replicating cells seen in more confluent cultures (Lee et al., Blood 107:2153-2161 (2006)).

Immunoselection can also be used to isolate hMSCs using monoclonal antibodies raised against surface antigens expressed by bone marrow-derived hMSCs. For example, U.S. Pat. No. 6,387,367 describes the use of monoclonal antibodies SH2, SH3 or SH4; the SH2 antibody binds to endoglin (CD105), while SH3 and SH4 bind CD73. A stro-1 antibody is described in Gronthos et al., 1996, J. Hematother. 5: 15-23. Further cell surface markers that may be used to enrich for human MSCs are described in Table I, page 237 of Fibbe et al., Ann. N.Y. Acad. Sci. 996: 235-244 (2003).

MSCs may be derived from any animal, including but not limited to a rodent, a horse, a cow, a pig, a dog, a cat, a non-human primate, and a human.

MSCs for use in the methods of the invention can be autologous, allogeneic or xenogeneic. The term “autologous” as used herein means that the transplant is derived from the cells, tissues or organs of the recipient. The term “allogeneic” as used herein means that the transplant is derived from cells, tissues, or organs that are of the same species as the recipient but antigenically distinct. The term “xenogeneic” as used herein means that the transplant is derived from the cells, tissues, or organs originating from a different species.

### B. Culture and Expansion of MSCs In Vitro

MSCs can be used immediately following isolation. Alternatively, MSCs can be transiently cultured, for example for 24 hours or less, prior to their use. MSCs can also be expanded in culture prior to their use in the methods of the invention.

In one embodiment of the methods described herein, MSCs are culture expanded to increase total cell numbers, prior to administering to the individual. Methods to expand MSCs in culture are described for example in U.S. Patent Publication Nos. 2004/0235166, 2005/0084494, and 2004/0235165, and U.S. Pat. No. 7,056,738.

MSCs may be frozen following isolation, and stored for any length of time that does not compromise their function, pluripotency or viability. MSCs can be frozen immediately after isolation, or cultured and expanded after isolation but prior to freezing. Frozen cells may then be thawed and used for the methods of the invention.

MSCs for use in the methods of the invention can be maintained in culture media which can be chemically defined serum free media or can be a “complete medium”, such as Dulbecco's Modified Eagles Medium supplemented with 10% serum (DMEM). Suitable chemically defined serum free media and complete media are well known in the art, see for example U.S. Pat. No. 5,908,782, WO96/39487, and U.S. Pat. No. 5,486,359. Chemically defined medium typically comprises a minimum essential medium such as Iscove's Modified Dulbecco's Medium (IMDM), supplemented with human serum albumin, human Ex Cyte lipoprotein, transferrin, insulin, vitamins, essential and non-essential amino acids, sodium pyruvate, glutamine and a mitogen. These media stimulate multipotent stromal cell growth without differentiation.

The invention also provides methods to culture the MSCs under conditions to remove any non-human serum proteins, prior to their administration to humans. Such methods include the use of short-term cultures in human serum or platelet lysate to metabolically remove non-human serum proteins (Yamada et al., 2004; Doucet et al., 2005; Spees et al., Molec. Ther. 9:747-756 (2004)).

In certain embodiments, MSCs can be genetically modified prior to administration to the individual. For example, the MSCs can be genetically modified to express a recombinant polypeptide, such as a growth factor, chemokine, or cytokine, or a receptor which binds growth factors, chemokines, or cytokines. The MSCs can also be genetically modified to express a marker protein such as GFP which allows their identification in the recipient.

### III. Methods of Administration

The MSCs term “transplanting” as used herein means introducing a cellular, tissue or organ composition into the body of a mammal by any method known in the art, or as indicated herein. The composition is a “transplant”, and the mammal is the recipient.

The transplant and recipient may be syngeneic, allogenic, or xenogeneic. The term “syngeneic” as used herein means that the transplant is derived from cells, tissues, or organs that are of the same species as the recipient, and antigenically the same or similar enough so as not to illicit an immune response, i.e., that are histocompatible. Syngeneic cells are sometimes referred to herein as “HLA compatible.” The term “allogeneic” as used herein means that the transplant is derived from cells, tissues, or organs that are of the same species as the recipient but antigenically distinct. The term “xenogeneic” as used herein means that the transplant is derived from the cells, tissues, or organs originating from a different species. In one embodiment, the MSCs are autologous. The term “autologous” as used herein means that the transplant is derived from the cells, tissues or organs of the recipient.

In one embodiment, the animal to which the multipotent stromal cells are administered is a mammal. The mammal may be a rodent, a horse, a cow, a pig, a dog, a cat, a non-human primate, and a human.

The multipotent stromal cells can be administered to the individual by a variety of procedures. The multipotent stromal cells may be administered systemically, such as by intravenous, intraarterial, or intraperitoneal administration, or the multipotent stromal cells may be administered directly to a tissue or organ such as the pancreas or kidney, for example by direct injection into the tissue or organ.

The MSCs are administered to the individual in a therapeutically effective amount, as described above. In general, the MSCs are administered in an amount of from about 1×10⁵ cells/kg to about 1×10⁷ cells/kg. The exact amount of MSCs to be administered is dependent upon a variety of factors, including the age, weight, and sex of the patient, and the extent and severity of the condition being treated.

The MSCs may be administered in conjunction with an acceptable pharmaceutical carrier. For example, the MSCs may be administered as a cell suspension in a pharmaceutically acceptable liquid medium for injection.

It is to be understood that the multipotent stromal cells, when employed in the above-mentioned therapies and treatments, may be administered in combination with other therapeutic agents known to those skilled in the art. In one embodiment, the recipient can be administered an agent that suppresses the immune system, such as Tacrolimus, Sirolimus, cyclosporine, and cortisone and other drugs known in the art. See e.g. U.S. Patent Publication No. 2004/0209801. Other immunosuppressive agents which can be used include anti-CD11 antibody.

The following examples provide illustrative embodiments of the invention. One of ordinary skill in the art will recognize the numerous modifications and variations that may be performed without altering the spirit or scope of the present invention. Such modifications and variations are encompassed within the scope of the invention. The Examples do not in any way limit the invention.

### EXAMPLES

### Example 1

### Human Multipotent stromal Cell (MSCs) from Marrow Promote Regeneration of Insulin-Producing Islets in Diabetic NOD/scid Mice

For treating diabetes, some of the most attractive candidates are the plastic adherent cells which can be isolated from human marrow, referred to variously as colony-forming unit fibroblastic, multipotent stromal cells, mesenchymal stem cells, multipotential stromal cells, or MSCs (Owen and Friedenstein, 1988; Caplan, 1991; Prockop, 1997). MSCs are readily obtained from a patient, and rapidly expanded in culture so that it is feasible to administer very large numbers of autologous cells to patients. After systemic infusion, the cells can home to injured tissues and repair them by several different mechanisms, including differentiating into multiple cellular phenotypes, providing cytokines and chemokines, enhancing the proliferation of tissue-endogenous stem/progenitor cells, or cell fusion or transfer of mitochondria (Prockop et al., 2003; Spees et al., 2003; Munoz et al., 2005; Spees et al., 2006). In addition, MSCs suppress some immune reactions (Le Blanc et al., 2003). A further attractive feature of MSCs is that they have been tested in clinical trials for severe forms of osteogenesis imperfecta (Horwitz et al., 2001), mucopolysaccharidoses (Koc et al., 2002), and graft versus host diseases (Lazarus et al., 2005; Le Blanc et al., 2004). These individual trials have provided promising results, without any apparent toxicity in patients.

We elected to test the effectiveness of MSCs from human bone marrow (hMSCs) in immunodeficient NOD/scid in which moderately severe diabetes was produced with streptozotocin (STZ). The strategy made it possible to readily detect and assay the effectiveness of the donor human cells without the use of exogenous labels that might provide artifactual results.

### Materials and Methods

STZ Induced Diabetes in Mice: Male immune-deficient NOD/scid mice (NOD.CB17-Prkdc^{scid}/J; Jackson Laboratories, Bar Harbor, Me.) 7 to 8 weeks of age were injected intraperitoneally (IP) with 35 mg/kg STZ (Sigma-Aldrich; St. Louis, Mo.) daily on Days 1 to 4. STZ was solubilized in sodium citrate buffer, pH 4.5, and injected within 15 minutes of preparation. The mice were maintained under sterile conditions under protocols approved by the Institutional Animal Care and Utilization Committees of the Tulane University and the Ochsner Clinic Foundation.

Preparation and Infusion of hMSCs: Frozen vials of hMSCs from passage 2 were obtained from the Tulane Center for the Preparation and Distribution of Adult Stem Cells (http://www.som.tulane.edu/gene_therapy/distribute.shtml). The cells were prepared as described (Sekiya et al., 2002) from normal volunteers with protocols approved by an Institutional Review Board. The frozen vials of about 10⁶ passage 1 human MSCs were thawed, plated in 25 ml medium in a 180 cm² culture plate (Nunc) in complete culture medium containing 20% fetal calf serum (Sekiya et al., 2002), and incubated at 37° C. with 5% humidified CO₂. After 24 hours, the medium was removed, adherent viable cells were washed twice with PBS, harvested with 0.25% trypsin and 1 mM EDTA at 37° C. for about 5 minutes, and replated at 100 cells/cm². The cells were incubated for 7 to 9 days until they were 70% confluent, at which time they were harvested with trypsin/EDTA. For transplantation, the cells were washed by centrifugation with PBS, suspended in Hank's Balanced Salt Solution at a concentration of 20,000 cells per μl, and maintained at 4° C. Mice were anesthetized IP with 0.07 ml mixture of ketamine (91 mg/kg) and xylazine (9 mg/kg), and 150 μl of cell suspension were injected through the chest wall into the left ventricle.

Assays for Blood Glucose and Insulin: Blood glucose was assayed in tail vein blood with a glucometer (Elite Diabetes Care System; Bayer, Germany) after a four hour morning fast. Blood insulin was assayed on blood obtained by intracardiac puncture of anesthetisized mice before sacrifice on Day 32 using both a mouse-specific ELISA kit and a human-specific ELISA kit (Ultrasensitive Mouse Insulin'ELISA, and Insulin Ultrasensitive ELISA; Mercodia, Uppsala, Sweden).

Preparation of Tissue Samples: Mice were sacrificed by IP injection of ketamine/xylazine, and perfused through the left ventricle with 20 ml of PBS and then through the right ventricle with 5 ml of PBS before tissues were isolated by dissection. The distal half of pancreas, one kidney, and other organs were rapidly frozen at −80° C. for DNA and RNA assays. The proximal half of pancreas was fixed overnight in 10% buffered formalin, and incubated overnight at 4° C. in 30% sucrose/PBS. The samples were then embedded in a gel (Tissue-Tek Oct Compound; Sakura Finetek, Torrance, Calif.) to prepare frozen sections 5 to 8 μm. Samples of kidney for histology were fixed with the same protocol and used to prepare parafin sections of 8 μm. Samples of kidney for immunohistology were embedded in the gel and used to prepare frozen sections of 8 to 30 μm.

Real Time PCR Assays and RT-PCR Assays: Frozen tissues were homogenized, DNA extracted with phenol/chloroform (Phase Lock Gel; Eppendorf/Brinkmann Instruments, Inc., Westbury, N.Y.), and total DNA assayed by absorbance. Real time PCR assay was performed with 200 ng of target DNA, Alu-specifc primers and a fluorescent probe (McBride et al., 2003) using an automated instrument (Model 7700; Applied Biosystems, Foster City, Calif.). Values for the amount of target DNA in each sample were corrected by assays for the single copy mouse albumin gene (Lee et al., 2006).

For RT-PCR assays, RNA was isolated from distal portion of mouse pancreas (RNeasy RNA Isolation Kit; Qiagen, Valencia, Calif.). As a control, RNA from human pancreas was obtained from a commercial source (Clontech; Mountain View, Calif.). Approximately 100 ng total RNA was used for cDNA synthesis by reverse transcriptase (M-MLV RT Kit; Invitrogen, Carlsbad, Calif.). The samples were incubated at 37° C. for 50 minutes followed by 15 minutes at 70° C. to inactivate the reverse transcriptase. The cDNAs were amplified by PCR (Recombinant Taq DNA polymerase; Invitrogen, Carlsbad, Calif.) with 30 cycles at 94° C. for 30 seconds, 60° C. for 30 seconds, and 72° C. for 30 seconds. PCR primers were human insulin forward: 5′-AGC CTT TGT GAA CCA ACA CC-3′ (SEQ ID NO: 1); and human insulin reverse: 5′-TCC GCC AAA ATA ACC GAT GTG AT-3′ (SEQ ID NO: 2). Samples were separated on a 2% agarose gel with or without prior cleavage with SbfI or EcoNI (New England Biolabs, Ipswich, Mass.). To correct for the efficiency of reverse transcription, samples were also assayed for human GAPDH mRNA with forward primer: 5′-TCA ACG GAT TTG GTC GTA TTG GG-3′ (SEQ ID NO: 3); reverse: 5′-TGA TTT TGG AGG GAT CTC GC-3′ (SEQ ID NO. 4); and for mouse GAPDH mRNA with forward primer: 5′-CGT CCC GTA GAC AAA ATG GT-3′ (SEQ ID NO: 5); and reverse: 5′-TTC CCA ITT TCA GCC TTG AC-3′ (SEQ ID NO: 6).

Histology and Morphology: For histology of pancreas, sections were stained with hematoxylin/eosin. For histology of kidney, sections were stained with periodic acid-Schiff (PAS, Richard-Allan Scientific, Kalamazoo, Mich.). For immunohistochemistry, frozen sections were incubated for 18 hours at 4° C. with primary antibodies to a anti-human β2-microglobulin (1:200; Roche, Switzerland), anti-human nuclei antigen (1:200; Chemicon, Temecula, Calif.), anti-human insulin (1:40; Calbiochem, San Diego, Calif.), anti-mouse insulin (1:50; R&D Systems, Minneapolis, Minn.), anti-mouse/human PDX-1 (1:50; R&D Systems), anti-mouse/human podocalyxin (1:100; R&D Systems), anti-mouse macrophages/monocytes (1:25, Chemicon), anti-mouse/human fibronectin (1:80; Chemicon, Temecula, Calif.) or anti-mouse/human CD31 (1:500; BD Biosciences, San Jose, Calif.). Slides were washed three times for 5 minutes with PBS and incubated for 45 minutes at room temperature with species-specific secondary antibodies (1:1000; Alexa-594 or Alexa-488; Molecular Probes, Eugene, Oreg.). Controls included omitting the primary antibody. Slides were evaluated by epifluorescence microscopy (Eclipse E800; Nikon, Melville, N.Y.). A Leica DMRXA microscope equipped with an automated x, y, z stage and CCD camera (Sensicam, Intelligent Imaging Innovations, Denver, Colo.) was used for image deconvolution. Images taken at 0.4 μm intervals were deconvoluted using commercial software (Slidebook Software, Intelligent Imaging Innovations, Denver, Colo.).

Urine Assays: Mice on Day 39 to Day 45 were placed in individual metabolic cages (NALGENE Labware, Rochester, N.Y.) and 18 hour urine samples were assayed for albumin (Quantichrom™ BCG Albumin Assay Kit; Bioassay Systems, Haywood, Calif.).

### Results

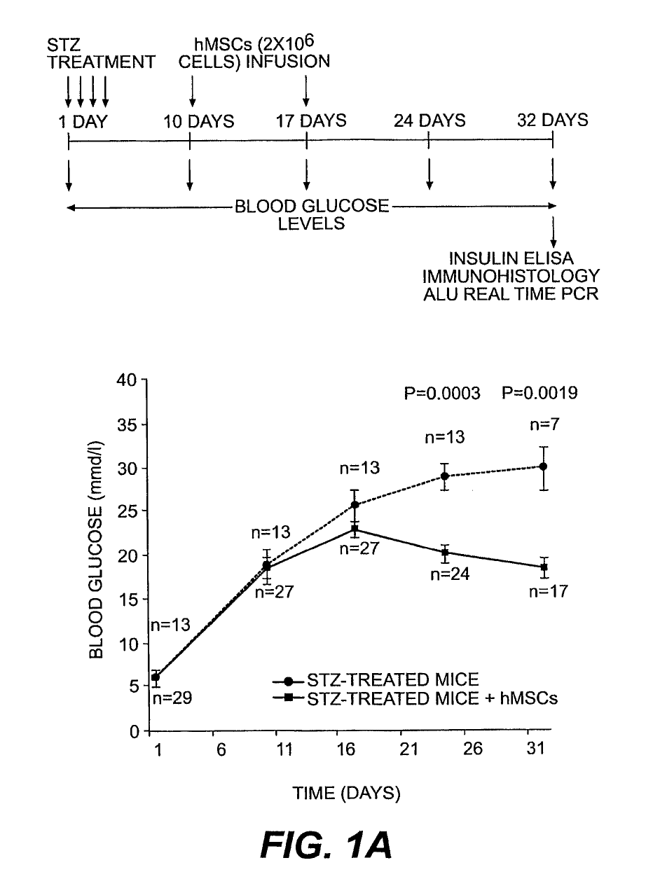

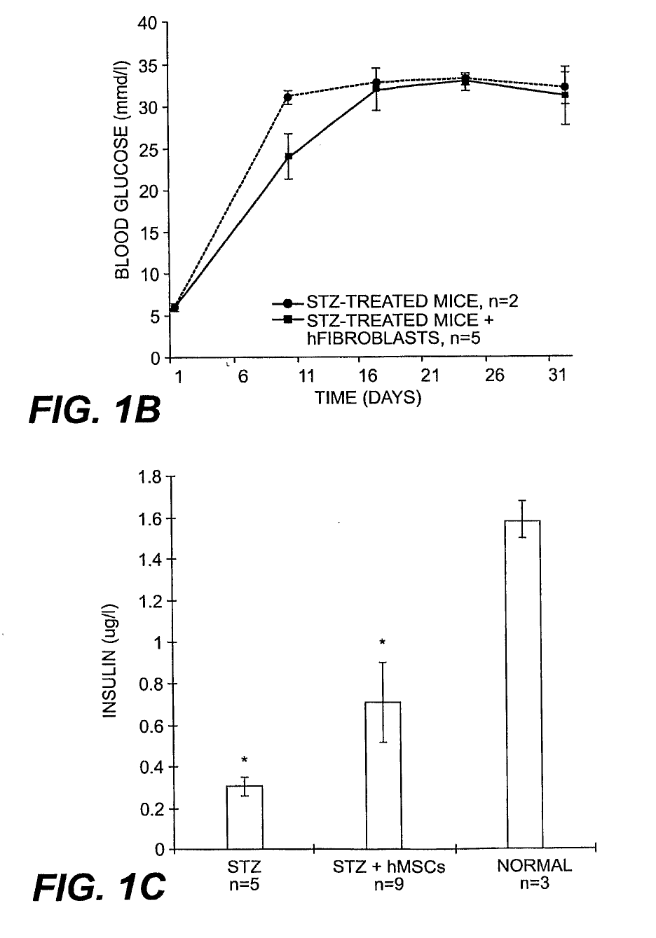

The Diabetic Model. STZ was used to produce diabetes in NOD/scid mice. The mice do not spontaneously develop diabetes but lack functional B and T cells and have lymphopenia and hypogammaglobulimia together with a normal hematopoietic microenvironment (Serreze et al., 1995). Multiple low doses of STZ were administered to the mice (FIG. 1, top panel) under conditions that tend to minimize nephrotoxicity from the drug (Tay et al., 2005). In initial experiments, we administered 35 mg/kg STZ daily for 5 days following the protocol of Hess et al. (2003), but the mice either died or had to be sacrificed after 3 to 5 weeks because of severe weight loss and cachexia. Therefore we reduced the dose to 35 mg/kg for 4 days only. With the 4-day regimen, blood glucose levels increased from normal levels (5.92 mM+/−0.98 S.E.) to severe hyperglycemic levels (FIG. 1A), but the mice survived for over 1 month without administration of insulin. The diabetic mice weighed less than controls (24.03 g+/−3.13 S.D. vs. 27.83 g+/−1.65 S.D.; n=5, p=0.02). Also, the diabetic mice had a marked increase in urinary volume at Days 39 to 45 (5.04 ml+/−3.18 S.D. vs. 0.44 ml+/−0.3 S.D.; n=7, p=0.005). None of the mice however developed albuinuria.

Infusion of hMSCs Lowered Blood Sugar and Increased Blood Insulin.

About 2.5×10⁶ hMSCs were infused into the diabetic mice on Day 10 and again on Day 17. To avoid aggregation of the hMSCs and to ensure reproducible delivery, the hMSCs were suspended in a large volume of buffer (150 μl) at a concentration of about 17,000 cells/μl and injected through the chest wall into the left ventricle. The blood glucose levels in the hMSC-treated diabetic mice decreased significantly by Day 24 and Day 32 (p=0.0003 and 0.0019, respectively; FIG. 1A). There was no difference between untreated diabetic mice and hMSC-treated diabetic mice in body weight (23.7 g+/−2.37 S.D.; n=15), but there was a reduction in urinary volume (2.20 ml+/−3.3 S.D.; n=7 vs. 5.04 ml+/−3.18 S.D.; n=7, p=0.029). Human skin fibroblasts infused into the diabetic mice under the same conditions had no effect on blood glucose levels (FIG. 1B).

ELISA assays on blood demonstrated that the administration of the hMSCs to the diabetic mice increased the levels of circulating mouse insulin (0.70 μg/L+/−0.11 S.D. vs. 0.30 μg/L+/−0.04 S.D.; n=5 or 9; p=0.0018; FIG. 1C). Assays of the same samples were negative for human insulin (not shown).

Detection of Human DNA from hMSCs in Pancreas and Kidney of Diabetic NOD/scid Mice. Tissues from the hMSC-treated diabetic mice were assayed for engraftment by real time PCR assays for human Alu sequences (McBride et al., 2003). In 9 of 13 mice, human DNA equivalent to from 0.11 to 2.9% of the DNA infused as hMSCs was detected in the pancreas on Day 17 or Day 32 (Table 1). In 4 of the 13 mice, no human genomic DNA was detected on Day 32, perhaps because of the technical difficulty in consistently injecting cells into the left ventricle. In 6 mice in which human DNA was detected in the pancreas, human DNA was also detected in kidney (Table 1). In 4 of the 6 mice, the recovery of human DNA in kidney was unusually high and accounted for 6.7 to 11.6% of the human DNA infused as hMSCs. Variable amounts of human DNA (equivalent to 0 to 0.22% of the infused DNA) were also detected in the hearts of mice into which the hMSCs were infused (not shown). Human Alu sequences were not detected in lung, liver and spleen. Human Alu sequences also were not detected in any of the same tissues 22 days after infusion of cultured human fibroblasts (Table 1).

TABLE 1
Engraftment assayed by Real Time PCR for Alu.

| Animal/cells | Days | Pancreas | Kidney |
|---|---|---|---|
| 1 hMSC | 17 | 2.95 ± 0.06 | 6.70 ± 0.06 |
| 2 hMSC | 32 | 1.02 ± 0.31 | 0.05 ± 0.004 |
| 3 hMSC | 32 | 0.78 ± 0.05 | 11.58 ± 2.16 |
| 4 hMSC | 32 | 0.22 ± 0.03 | 0.03 ± 0.05 |
| 5 hMSC | 32 | 0.07 ± 0.01 | 10.62 ± 0.715 |
| 6 hMSC | 32 | 0.04 ± 0.02 | 9.82 ± 1.23 |
| 7 hMSC | 32 | 0.36 ± 0.02 | NA* |
| 8 hMSC | 32 | 0.19 ± 0.09 | NA* |
| 9 hMSC | 32 | 0.11 ± 0.01 | NA* |
| 10-13 hMSC | 32 | ND** | ND** |
| 14-18 hFibro*** | 22 | ND** | ND** |

NA* Not assayed.
ND** Not detected.
hFibro*** Human skin fibroblasts. Tissues assayed 22 days after infusion.
Values are % of human DNA infused as cells.

Increased Pancreatic Islets in hMSC-treated Diabetic Mice.

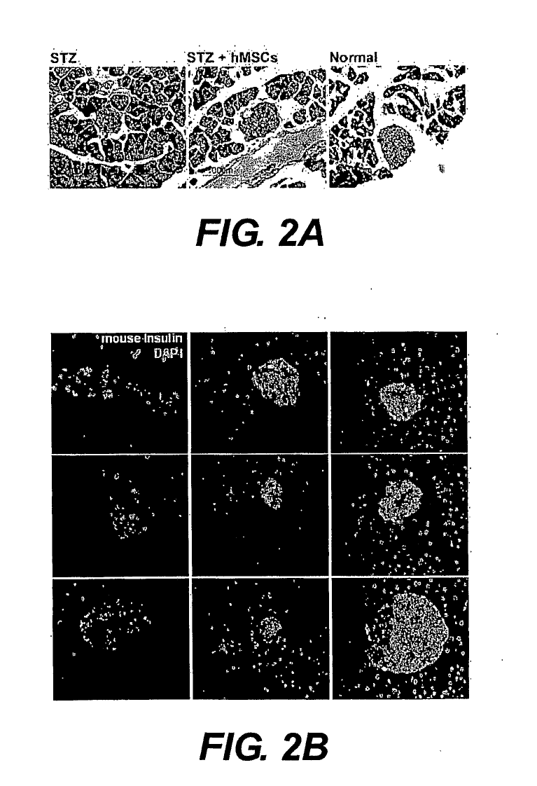

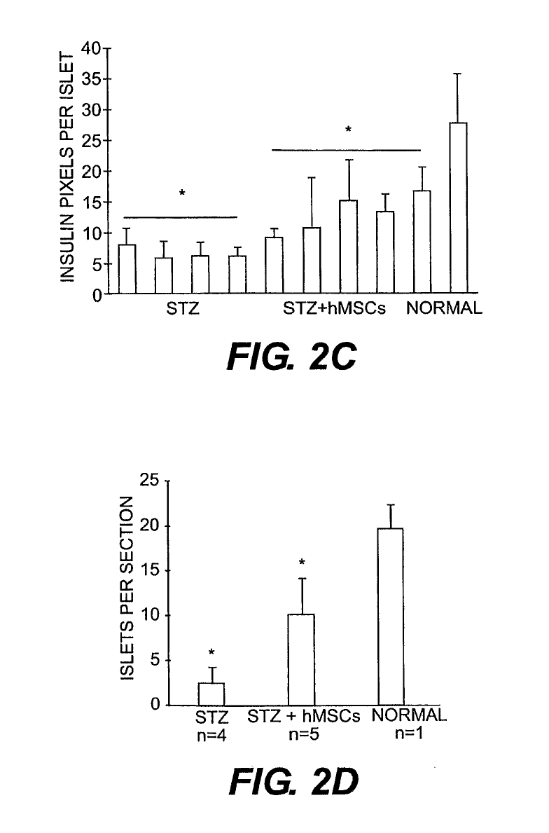

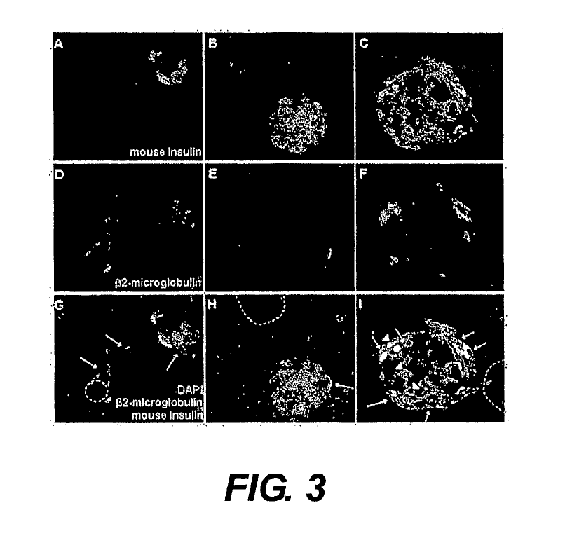

Tissues with high levels of human Alu sequences were selected for microscopy. Pancreases from the STZ-diabetic mice revealed smaller islets (FIG. 2A). They had a decrease in mouse insulin content as assayed by labeling with antibodies (FIGS. 2B and 2C), and a decreased number of islets per section (FIG. 2D). In pancreases from hMSC-treated diabetic mice, the islets appeared larger compared to islets from untreated diabetic mice (FIG. 2A). Also, the islets had an increase in mouse insulin as assayed by labeling with antibodies (FIGS. 2B and 2C), and there was an increase in number of islets per section (FIG. 2D). Many of the islets in the hMSC-treated diabetic mice appeared to bud off the pancreatic ducts (FIGS. 2A and 3).

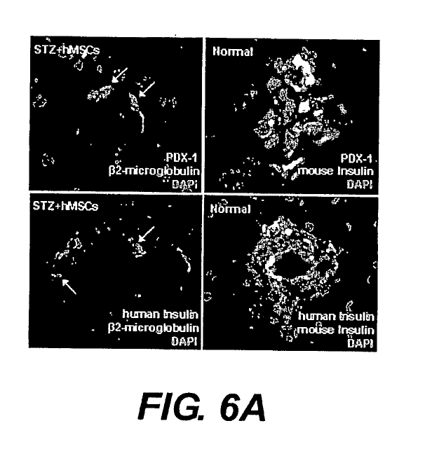

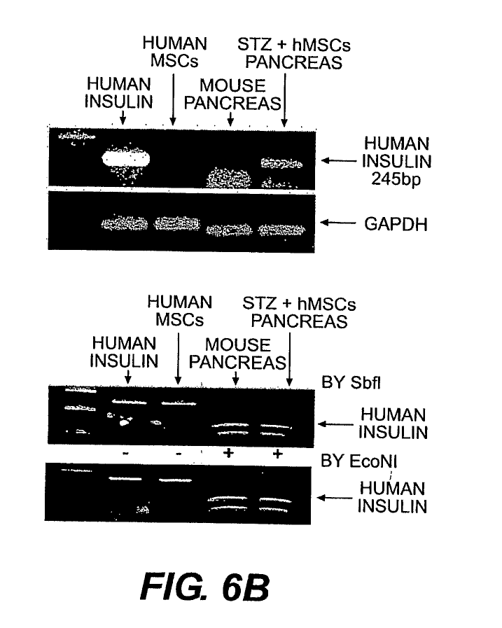

Small numbers of human cells were detected in islets of the hMSC-treated diabetic mice by labeling sections with antibodies to human β2-microglobulin and mouse insulin (FIG. 3). A few of the cells labeled for human β2-microglobulin co-labeled with a human-specific antibodies both to PDX-1 and human insulin (FIG. 6A). Qualitative RT-PCR assays of RNA from the pancreas of one hMSC-treated diabetic mouse detected mRNA for human insulin (FIG. 6B). However, samples from 11 additional hMSC-treated diabetic mice were negative both by immunolabeling and RT-PCR assays for human insulin.

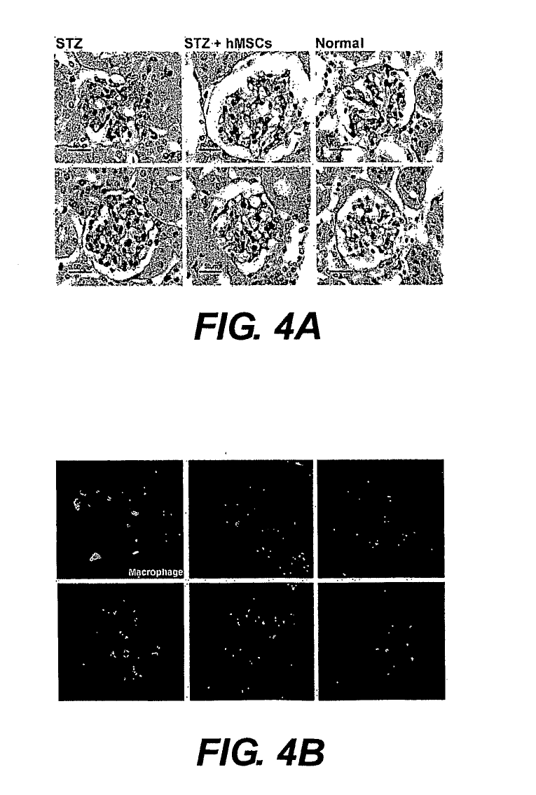

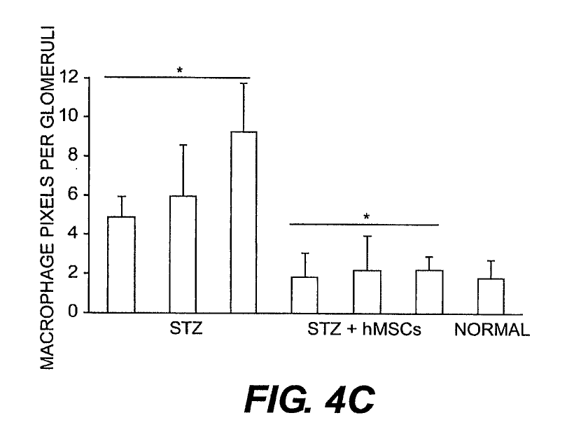

Glomerular Morphology in hMSC-treated Diabetic Mice. Kidneys from untreated diabetic mice at Day 32 contained many abnormal glomeruli with increased deposits of extracellular matrix protein in mesangium (FIG. 4A). In kidneys from hMSC-treated diabetic mice that had high levels of human Alu sequences, glomeruli were more normal in appearance. The differences were accentuated by labeling kidney sections with antibodies to mouse macrophages/monocytes (FIGS. 4B and 4C). In the untreated diabetic mice, there was a marked increase in macrophages in the glomeruli; few were seen in the glomeruli from the hMSC-treated diabetic mice.

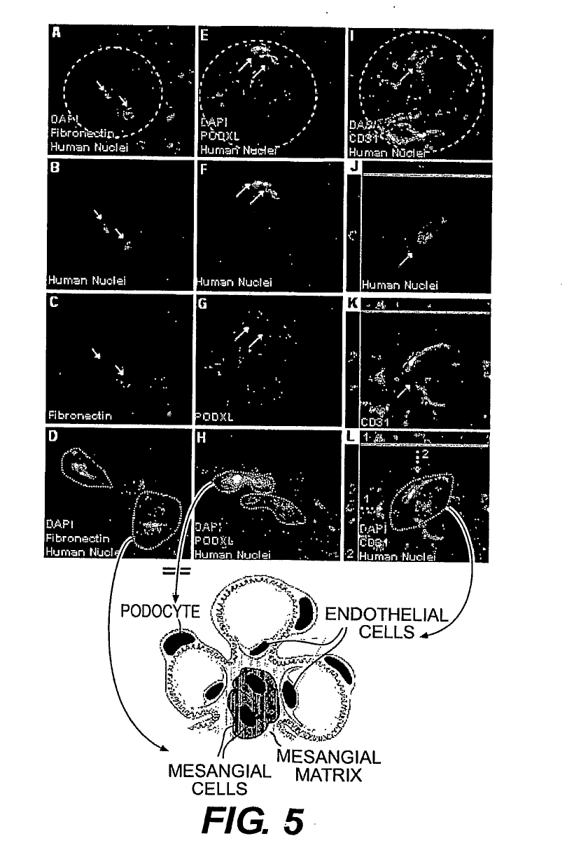

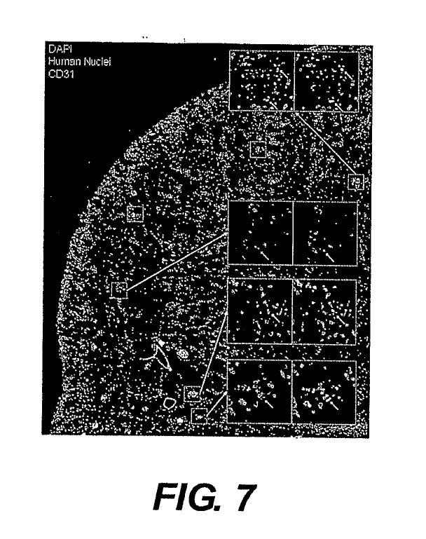

Kidneys that showed high levels of engraftment of human Alu sequences (Table 1) were also assayed for human cells. Frozen sections labeled with antibodies to human nuclei antigen demonstrated that human cells were present in the glomeruli of hMSC-treated diabetic mice (FIGS. 5, 7, and 8). In some sections, human cells were present in about one-fifth of the glomeruli (FIG. 7), an observation consistent with the PCR assays for human Alu sequences (Table 1). Human cells were not found in tubules. Most positive glomeruli had one human cell. Glomeruli with two or more human cells were rare and in such glomeruli, the human cells were usually widely dispersed. These results indicate that the human cells had not propagated after engrafting in kidney.

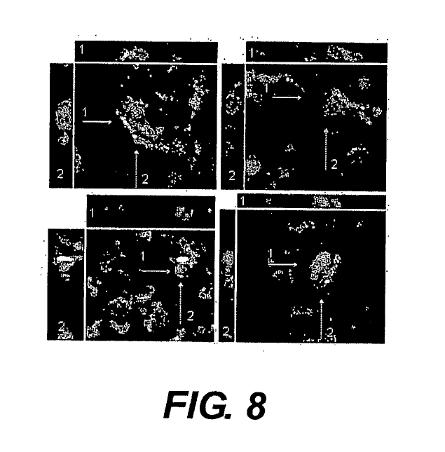

Double immunohistochemistry suggested that some of the human cells were also labeled with a monoclonal antibody to CD31 (PECAM-1), an endothelial cell membrane epitope (FIGS. 5I-L, 7, and 8). CD31 was not expressed in cultured hMSCs (not shown). Also, in some sections in which the cells were captured in the appropriate orientation, the human cells that expressed CD31 had the elongated morphology of endothelial cells (FIG. 5L and FIG. 8). These results indicate that some of the human cells differentiated into endothelial cells. Some of the human cells also expressed fibronectin (FIGS. 5A-5L), a protein expressed in mesangial cells. The co-labeled cells had the rounded morphology of mesangial cells. However, fibronectin was also expressed in cultured hMSCs and therefore it was not clear whether the cells had differentiated into mesangial cells. No cells were found that co-labeled with antibodies to human nuclei antigen and podocalyxin, a protein expressed in podocytes (FIGS. 5E-5H).

Two aspects of the observations made here are remarkable: The selective homing of hMSCs to both pancreatic islets and renal glomeruli of the diabetic mice, and the ability of the cells to repair the tissues.

The results obtained here indicate that up to 3% of the infused hMSCs engrafted into pancreas and up to 11% of the infused cells engrafted into kidney in the diabetic mice (Table I). Previous reports demonstrated only very low levels of engraftment after systemic infusion MSCs into uninjured adult rodents (Lee et al., 2006). Intracardiac infusion instead of intravenous infusion of the cells probably decreased trapping of the cells in the capillary beds of the lung, but it was apparent that the highest levels of engraftment were seen in the two organs damaged in the diabetic model; significant numbers of cells were not detected in lung, liver or spleen. The cells in the renal glomeruli were single cells, an observation suggesting that they engrafted immediately after systemic infusion into the mice, probably in response to specific signals from the injured tissues.

The infused hMSCs improved the hyperglycemia and increased blood levels of mouse insulin in the diabetic mice. Some of the human cells that engrafted into the pancreas differentiated so as to express both PDX-1 and human insulin. However, the major effect of the hMSC treatment was to increase the number of mouse islets and mouse insulin-producing cells. In the treated diabetic mice, new islets appeared to bud off pancreatic ducts that are the source of islets during early development of the pancreas (Hardikar et al., 2004). These observations are similar to the recent observations that hMSCs implanted into the dentate gyrus of the hippocampus of immunodeficient mice enhanced proliferation, migration and neural differentiation of the nearby endogenous mouse neural stem cells (Munoz et al., 2005).

The engraftment of the hMSCs into kidney was associated with improvements in glomerular morphology, a decrease in mesangial thickening, and a decrease in macrophage infiltration. STZ is a DNA alkylating reagent and single large doses produce tubular necrosis, but repeated lower doses and the resulting hyperglycemia produce glomerular changes more typical of but not identical to diabetic nephropathy (Tay et al., 2005). The observations here do not eliminate the possibility that the improvements in the glomeruli were secondary to the lower blood glucose levels in the treated diabetic mice. However, it was striking that the human cells were found exclusively in the glomeruli, and that some the cells differentiated into cells with characteristics of endothelial cells. These results demonstrate that administration of hMSCs improved the renal lesions, either by preventing the pathological changes in the glomeruli or enhancing their regeneration.

The observations presented here demonstrate that hMSCs may be useful to treat both the hyperglycemia and the renal damage associated with hyperglycemia seen in diabetic patients. Autologous hMSCs are readily generated in a few weeks from patients (Sekiya et al., 2002), and risks from administration of autologous hMSCs to patients are thought to be relatively minimal. MSCs or related cells from bone marrow have been shown to produce beneficial effects in animal models for a variety of diseases and in several clinical trials, including clinical trials in heart disease that are now being conducted at multiple medical centers (Prockop et al., 2003; Ye et al., 2006; Fazel et al., 2005). Systemic infusion of autologous hMSCs in patients with diabetes could also have beneficial effects in several of the many tissues damaged by the disease.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patents, and biological sequences cited in this disclosure are incorporated by reference in their entirety. To the extent the material incorporated by reference contradicts or is inconsistent with the present specification, the present specification will supersede any such material. The citation of any references herein is not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

### REFERENCES

Aggarwal et al., Blood 105:1815-1822 (2005).
Baddoo et al., J. Cell Biochem. 89:1235-1249 (2003).
Banerjee et al., Biochem. Biophys. Res. Commun. 328:318-325 (2005).
Caplan, J. Orthop. Res. 9:641-650 (1991).
Chen et al., World J. Gastroenterol. 10:3016-3020 (2004).
Choi et al., Biochem. Biophys. Res. Commun. 330:1299-1305 (2005).
Ende et al., Biochem. Biophys. Res. Commun. 321:168-171 (2004).
Fazel et al., Ann. Thorac. Surg. 79:52238-52247 (2005).
Hardikar, Trends Endocrinol. Metab. 15:198-203 (2004).
Hess et al., Nat. Biotechnol. 21:763-770 (2003).
Horwitz et al., Blood 97:1227-1231 (2001).
Ianus et al., J. Clin. Invest. 111:843-850 (2003).
Kang et al., Exp. Hematol. 33:699-705 (2005).
Koc et al., Bone Marrow Transplant 30:215-222 (2002).
Lazarus et al., Biol. Blood Marrow Transplant. 11:389-398 (2005).
Le Blanc et al., Scand. J. Immunol. 57:11-20 (2003).
Le Blanc et al., Lancet. 363:1439-1441 (2004).
Lechner et al., Diabetes 53:616-623 (2004).
Lee et al., Blood 107:2153-2161 (2006).
McBride et al., Cytotherapy 5:7-18 (2003).
Munoz et al., Proc. Natl. Acad. Sci. U.S.A. 102:18171-18176 (2005).
Oh et al., Lab Invest. 84:607-617 (2004).
Owen et al., Ciba Found. Symp. 136:42-60 (1998).
Peister et al., Blood 103:1662-1668 (2004).
Pittenger et al., Science 284:143-147 (1999).
Prockop et al., Proc. Natl. Acad. Sci. U.S.A. 100:11917-11923 (2003).
Rubio et al., Cancer Res. 65:3035-3039 (2005).
Sekiya et al., Proc. Natl. Acad. Sci. U.S.A. 99:4397-4402 (2002).
Serreze et al., Diabetes 44:1392-1398 (1995).
Spees et al., Proc. Natl. Acad. Sci. U.S.A. 103:1283-1288 (2006).
Spees et al., Proc. Natl. Acad. Sci. U.S.A. 100:2397-2402 (2003).
Taneera et al., Diabetes 55:290-296 (2006).
Tay et al., Kidney Int. 68:391-398 (2005).
Ye et al., Exp. Biol. Med. 231:8-19 (2006).

What is claimed is:

1. A method of treating diabetes in an individual comprising administering to said individual a therapeutically effective amount of isolated multipotent stromal cells.

2. The method of claim 1, wherein the administration of the multipotent stromal cells enhances regeneration of pancreatic islets in the individual.

3. The method of claim 1, wherein the administration of the multipotent stromal cells reduces hyperglycemia in the individual.

4. The method of claim 1, wherein the administration of the multipotent stromal cells increases insulin levels in the individual.

5. The method of claim 1, wherein the administration of the multipotent stromal cells improves the diabetic nephropathy in the individual.

6. The method of claim 1, wherein the multipotent stromal cells are isolated from a tissue selected from the group consisting of bone marrow, peripheral blood, umbilical cord blood, and synovial membrane.

7. The method of claim 6, wherein the multipotent stromal cells are isolated from bone marrow.

8. The method of claim 1, wherein the multipotent stromal cells are cultured in vitro prior to administration to the individual.

9. The method of claim 8, wherein the multipotent stromal cells are expanded in vitro prior to administration to the individual.

10. The method of claim 1, wherein the multipotent stromal cells are autologous.

11. The method of claim 1, wherein the multipotent stromal cells are allogeneic.

12. The method of claim 1, wherein the multipotent stromal cells are HLA compatible with the individual.

13. The method of claim 1, wherein the multipotent stromal cells are isolated from a mammal.

14. The method of claim 13, wherein the mammal is selected from the group consisting of a rodent, a horse, a cow, a pig, a dog, a cat, a non-human primate, and a human.

15. The method of claim 14, wherein the mammal is a human.

16. The method of claim 1, wherein the individual is a mammal.

17. The method of claim 16, wherein the mammal is selected from the group consisting of a rodent, a horse, a cow, a pig, a dog, a cat, a non-human primate, and a human.

18. The method of claim 17, wherein the mammal is a human.

19. The method of claim 1, wherein said administration is by infusion.

20. The method of claim 19, wherein said infusion is selected from the group consisting of intravenous infusion, systemic infusion, intra-arterial infusion, intracoronary infusion, and intracardiac infusion.

21. A method for preventing or inhibiting the progression of a diabetic complication in an individual in need thereof, comprising administering to said individual a therapeutically effective amount of isolated multipotent stromal cells.

22. The method of claim 21, wherein the diabetic complication is a diabetic microvascular complication.

23. The method of claim 22, wherein the diabetic microvascular complication is diabetic nephropathy.

24. The method of claim 22, wherein the diabetic microvascular complication is diabetic neuropathy.

25. The method of claim 22, wherein the diabetic microvascular complication is diabetic retinopathy.

26. The method of claim 21, wherein the diabetic complication is a cardiovascular disease.

27. The method of claim 21, wherein the multipotent stromal cells are isolated from a tissue selected from the group consisting of bone marrow, peripheral blood, umbilical cord blood, and synovial membrane.

28. The method of claim 27, wherein the multipotent stromal cells are isolated from bone marrow.

29. The method of claim 21, wherein the multipotent stromal cells are cultured in vitro prior to administration to the individual.

30. The method of claim 29, wherein the multipotent stromal cells are expanded in vitro prior to administration to the individual.

31. The method of claim 21, wherein the multipotent stromal cells are autologous or allogeneic.

32. A method for reversing hyperglycemia in an individual in need thereof,
comprising administering to said individual a therapeutically effective amount of isolated multipotent stromal cells.

33. The method of claim 32, wherein the hyperglycemia in the individual is caused by diabetes.

34. The method of claim 32, wherein the multipotent stromal cells are isolated from a tissue selected from the group consisting of bone marrow, peripheral blood, umbilical cord blood, and synovial membrane.

35. The method of claim 34, wherein the multipotent stromal cells are isolated from bone marrow.

36. The method of claim 32, wherein the multipotent stromal cells are cultured in vitro prior to administration to the individual.

37. The method of claim 36, wherein the multipotent stromal cells are expanded in vitro prior to administration to the individual.

38. The method of claim 32, wherein the multipotent stromal cells are autologous or allogeneic.

39. A method of reversing hypoinsulinemia in an individual in need thereof, comprising administering to said individual a therapeutically effective amount of isolated multipotent stromal cells.

40. The method of claim 39, wherein the hypoinsulinemia is caused by pancreatic damage.

41. The method of claim 40, wherein the pancreatic damage is caused by diabetes.

42. The method of claim 39, wherein the multipotent stromal cells are isolated from a tissue selected from the group consisting of bone marrow, peripheral blood, umbilical cord blood, and synovial membrane.

43. The method of claim 42, wherein the multipotent stromal cells are isolated from bone marrow.

44. The method of claim 39, wherein the multipotent stromal cells are cultured in vitro prior to administration to the individual.

45. The method of claim 44, wherein the multipotent stromal cells are expanded in vitro prior to administration to the individual.

46. The method of claim 39, wherein the multipotent stromal cells are autologous or allogeneic.

47. A method of enhancing the regeneration or repair of pancreatic islets in an individual in need thereof, comprising administering to the individual a therapeutically-effective amount of isolated multipotent stromal cells.

48. The method of claim 47, wherein the individual has diabetes.

49. The method of claim 47, wherein administration of the multipotent stromal cells increases the number of pancreatic islets in the individual.

50. The method of claim 47, wherein the administration of the multipotent stromal cells increases the size of the pancreatic islets in the individual.

51. The method of claim 47, wherein the multipotent stromal cells are isolated from a tissue selected from the group consisting of bone marrow, peripheral blood, umbilical cord blood, and synovial membrane.

52. The method of claim 51, wherein the multipotent stromal cells are isolated from bone marrow.

53. The method of claim 47, wherein the multipotent stromal cells are cultured in vitro prior to administration to the individual.

54. The method of claim 53, wherein the multipotent stromal cells are expanded in vitro prior to administration to the individual.

55. The method of claim 47, wherein the multipotent stromal cells are autologous or allogeneic.

56. The use of isolated multipotent stromal cells for treating diabetes in an individual in need thereof.

57. The use of isolated multipotent stromal cells in the manufacture of a medicament for treating diabetes in an individual in need thereof.
